(19) United States
(12) Patent Application Publication
(10) Pub. No.: US 2004/0193229 A1
(43) Pub. Date: Sep. 30, 2004

(54) Gastric electrical stimulation for treatment of gastro-esophageal reflux disease

(75) Inventors: Warren L. Starkebaum, Plymouth, MN (US); David A. Dinsmoor, St. Paul, MN (US)

Correspondence Address: MEDTRONIC, INC., 710 MEDTRONIC PARKWAY NE, MS-LC340, MINNEAPOLIS, MN, 55432-5604, US

(73) Assignee: Medtronic, Inc., Minneapolis, MN

(21) Appl. No.: 10/441,775

(22) Filed: May 19, 2003

Related U.S. Application Data
(60) Provisional application No. 60/381,634, filed on May 17, 2002.

(51) Int. Cl. A61N 1/18

(52) U.S. Cl. 607/40

(57) ABSTRACT

A system and method for treating gastro-esophageal reflux disease (GERD) in a patient are disclosed. Electrical stimulation pulses are generated by an implantable neurostimulator and delivered to a portion of a patient's stomach located downstream from the lower esophageal sphincter via an implantable medical electrical lead.

### RELATED APPLICATIONS

This application claims the benefit of the filing date and other benefits from U.S. Provisional Patent Application No. 60/381,634 to Starkebaum entitled “Gastric Electrical Stimulation for Treatment of Gastro Esophageal Reflux Disease” filed May 17, 2002, the entirety of which is hereby incorporated by reference herein. This application also hereby incorporates by reference herein in its entirety U.S. patent application Ser. No. ______ to Dinsmoor et al. entitled “Gastro-Electric Stimulation for Reducing the Acidity of Gastric Secretions or Reducing the Amounts Thereof” filed on even date herewith.

### FIELD OF THE INVENTION

The present invention relates to medical devices used to electrically stimulate the digestive system, and more specifically to devices employed to electrically stimulate portions of the stomach downstream from the lower esophageal sphincter and upstream from the pylorus to reduce the symptoms of gastro-esophageal reflux disease (GERD).

### BACKGROUND OF THE INVENTION

Conventional therapeutic approaches for gastro-esophageal reflux include numerous antacid and drug therapies; in cases where these approaches are unsatisfactory, a surgical procedure called fundoplication is sometimes employed.

U.S. Pat. No. 6,097,984 entitled “System and Method of Stimulation for Treating Gastro-Esophageal Reflux Disease” to Douglas, the entirety of which is incorporated herein by reference, describes a method for treating gastric reflux by electrical stimulation of the lower esophageal sphincter (LES). The Douglas system and method involves directly stimulating the LES of a patient in order to normally maintain it in a closed state. The stimulation is inhibited in response to patient swallowing, by monitoring esophageal motility and timing out an inhibition period following detection of motility representative of swallowing. The system utilizes an implanted stimulator which is programmed to deliver a train of stimulus pulses to one or more electrodes fixed around the gastro-esophageal junction and connected to the stimulator by one or more leads. The motility sensing is done by a sensor for sensing mechanical wave movement or electrical signals representative of high motility following swallowing. The motility sensor and stimulating electrodes are attached laproscopically, and are preferably carried by a common stent carrier which is sutured around the lower esophagus.

Some prior art publications relating to the present invention are listed below.

Kenneth Koch et al., “An Illustrated Guide To Gastrointestinal Motility,”
Electrogastrography, 2^{nd} Ed., pp. 290-307 (1993).
Kenneth Koch et al., “Functional Disorders of the Stomach,” Seminars in
Gastrointestinal Disease, Vol. 7, No. 4, 185-195 (October 1996).
Kenneth Koch, “Gastroparesis: Diagnosis and Management,” Practical
Gastroenterology (November 1997).
Babajide Familoni et al., “Efficacy of Electrical Stimulation at Frequencies
Higher than Basal Rate in Mayine Stomach,” Digestive Diseases and
Sciences, Vol. 42, No. 5 (May 1997).
Babajide O. Familoni, “Electrical Stimulation at a Frequency Higher than
Basal Rate in Human Stomach,” Digestive Diseases and Sciences, Vol.
42, No. 5 (May 1997).
Physician's Manual, NeuroCyberonics Prosthesis, Bipolar Lead, Model
300, September, 2001.
Schuster, Crowell and Koch, “Schuster Atlas of Gastrointestinal Motility
in Health and Disease,” 2^{nd} Ed., B.C. Decker, London (2002)
U.S. Pat. No. 5,188,104 to Wernicke et al. for “Treatment of Eating
Disorders by Nerve Stimulation.”
U.S. Pat. No. 5,231,988 to Wernicke et al. for “Treatment of Endocrine
Disorders by Nerve Stimulation.”
U.S. Pat. No. 5,263,480 to Wernicke et al. for “Treatment of Eating
Disorders by Nerve Stimulation.”
U.S. Pat. No. 5,292,344 to Douglas for “Percutaneously Placed
Electrical Gastrointestinal Pacemaker Stimulatory System, Sensing
System, and pH Monitoring System, With Optional Delivery Port.”
U.S. Pat. No. 5,423,872 to Cigaina for “Process and Device for Treating
Obesity and Syndrome Motor Disorders of the Stomach of a Patient.”
U.S. Pat. No. 5,540,730 to Terry for “Treatment of motility disorders by
nerve stimulation.”
U.S. Pat. No. 5,690,691 to Chen for “Gastro-intestinal pacemaker
having phased multi-point stimulation.”
U.S. Pat. No. 5,716,385 to Mittal for “Crural diaphragm pacemaker and
method for treating esophageal reflux disease.”
U.S. Pat. No. 5,836,994 to Bourgeois for “Method and apparatus for
electrical stimulation of the gastrointestinal tract.”
U.S. Pat. No. 5,925,070 to King et al. for “Techniques for adjusting the
locus of excitation of electrically excitable tissue.”
U.S. Pat. No. 5,941,906 to Barreras et al. for “Implantable, Modular
Tissue Stimulator.”
U.S. Pat. No. 6,083,249 to Familoni for “Apparatus for sensing and
stimulating gastrointestinal tract on-demand”
U.S. Pat. No. 6,097,984 to Douglas for “System and method of
stimulation for treating gastro-esophageal reflux disease.”
U.S. Pat. No. 6,238,423 to Bardy for “Apparatus and method for
treating chronic constipation.”
U.S. Pat. No. 6,381,496 to Meadows et al. for “Parameter context
switching for an implanted device.”
U.S. Pat. No. 6,393,325 to Mann et al. for “Directional programming for
implantable electrode arrays.”
U.S. Pat. No. 6,449,511 to Mintchev for “Gastrointestinal Electrical
Stimulator Having a Variable Electrical Stimulus.”
U.S. Pat. No. 6,453,199 to Kobosev for “Electrical Gastro-Intestinal
Tract Stimulator.”
U.S. Pat. No. 6,516,227 to Meadows et al. for “Rechargeable Spinal
Cord Stimulator System.”
U.S. patent application No. 09/537,070 to _{—————}for “_{——————”}
U.S. patent application Publication No. 2002 165589 for “Gastric
Treatment and Diagnosis Device and Method.”
U.S. patent application Publication No. 2003 014086 for “Method and
Apparatus for Electrical Stimulation of the Lower Esophageal Sphincter.”
U.S. patent application Publication No. 2002 116030 for “Electrical
stimulation of the Sympathetic Nerve Chain.”
U.S. patent application Publication No. 2002 193842 for “Heartburn and
Reflux Disease Treatment Apparatus.”
U.S. patent application Publication No. 2002 103424 for “Implantable
Medical Device Affixed Internally within the Gastrointestinal Tract.”
U.S. patent application Publication No. 2002 198470 for “Capsule and
Method for Treating or Diagnosing the Intestinal Tract.”
PCT patent application WO 0089655 for “Sub-Mucosal Gastric Implant
Device and Method.”
PCT patent application WO 0176690 for “Gastrointestinal Electrical
Stimulation.”
PCT patent application WO 02087657 for “Gastric Device and Suction
Assisted Method for Implanting a Device on a Stomach Wall.”
PCT patent application WO 0238217 for “Implantable Neuromuscular
Stimulator for the Treatment of Gastrointestinal Disorders.”

All patents and technical papers listed in Table 1 hereinabove are hereby incorporated by reference herein, each in its respective entirety. As those of ordinary skill in the art will appreciate readily upon reading the Summary of the Invention, Detailed Description of the Preferred Embodiments and Claims set forth below, at least some of the devices and methods disclosed in the patents and publications of Table 1 may be modified advantageously in accordance with the teachings of the present invention. The foregoing and other objects, features and advantages, which will now become more readily apparent by referring to the following specification, drawings and claims, are provided by the various embodiments of the present invention.

### SUMMARY OF THE INVENTION

In the present invention, electrical stimulation of appropriate portions of the stomach downstream from the lower esophageal sphincter reduces or treats symptoms attendant to GERD in a patient.

At least one electrical stimulation signal is applied to one or more appropriate portions of a patient's stomach at one or more locations situated downstream from the lower esophageal sphincter in an amount and manner effective to treat the symptoms of GERD. The at least one electrical stimulation signal is applied by an implantable neurological stimulator (INS) that has at least one medical electrical lead positionable, secured or attached to or in such location or in the vicinity thereof. Each such lead carries at least one electrode, and preferably at least two electrodes, positionable or attachable for contact with or in proximity to a suitable location in or near the patient's stomach downstream or below the lower esophageal sphincter. The electrical stimulation signal is provided in an amount and manner sufficient to treat or lessen the symptoms of gastro-esophageal reflux disease (GERD).

The present invention has certain objects. That is, various embodiments of the present invention provide solutions to one or more problems existing in the prior art respecting conventional treatment for GERD, including one or more of: (a) sequelae or side-effects resulting from the administration of pharmaceutical products; (b) the requirement to purchase expensive pharmaceutical products on an on-going basis; (c) when administering pharmaceutical products, not having the ability to terminate or change instantaneously administration of the therapy; and (d) lack of positive response to the administration of pharmaceutical therapy.

Various embodiments of the present invention have certain advantages, including one or more of: (a) targeted delivery of therapy; (b) ability to change the therapy delivered on-demand or instantaneously; (c) multiple methods of feedback control for optimizing therapy (e.g., pH, patient activated, time-dependent (e.g., activate stimulation therapy at mealtime); (d) lower cost than pharmaceuticals; (e) potential for the delivery of superior therapy; and (f) the patient does not have to remember to take a drug daily or according to a daily regimen.

Various embodiments of the present invention include one or more the features described below. Included among the various embodiments of the present invention is a therapy that would be used to treat gastro-esophageal reflux disease in cases, for example, where the patient does not respond to conventional drug therapy. The therapy could also be used in place of fundoplication surgery.

In one embodiment of the present invention, a system for treating gastro-esophageal reflux generally comprises a stimulator for generating stimulus pulses, and delivery means for delivering the stimulus pulses to the stomach downstream from the lower esophageal sphincter (e.g., adjacent the antrum or at another location, more about which we say below).

In another embodiment of the present invention, a gastro-electric stimulator for treating gastro-esophageal reflux in a patient generally comprises a neuro-electrical stimulator for producing a stimulation signal, and at least one electrical lead having a proximal end and a distal end. The proximal end of the electrical lead is connected to the neuro-electrical stimulator and the distal end is positionable in a lead position within the patient's abdomen. At least two electrodes are carried near the electrical lead distal end. The electrodes are electrically connected through the electrical lead to the neuro-electrical stimulator to receive the stimulation signal and convey this signal to an electrode position adjacent or within the stomach downstream of the lower esophageal sphincter (e.g., adjacent the antrum or in another suitable location).

Yet another embodiment of the present invention is a method for treating gastro-esophageal reflux in a patient. The method generally comprises diagnosing gastro-esophageal reflux in a patient; applying at least two electrodes to the stomach of the patient downstream of the lower esophageal sphincter; coupling the electrodes via at least one lead to a neurostimulator; and stimulating the digestive system with a stimulation signal generated by the neurostimulator and conveyed through the lead to the electrodes contacting the stomach of the patient downstream of the lower esophageal sphincter in an amount and manner to reduce or eliminate the symptoms of GERD in a patient.

In still another embodiment of the present invention, a method of treating gastro-esophageal reflux in a patient generally comprises diagnosing gastro-esophageal reflux in a patient; generating stimulus pulses; and delivering the stimulus pulses to the stomach downstream from the lower esophageal sphincter.

Such therapy may be effective for cases of severe reflux disease when drug therapies are ineffective. Compared to fundoplication, gastro-electrical stimulation (GES) is relatively less invasive, non-ablative, and reversible. It is anticipated that the side effects and complications from GES will be fewer compared to fundoplication. Fundoplication sometimes results in damage to the vagus nerve, which in turn may cause gastroparesis.

The gastric stimulation system of the present invention may also be employed to treat symptoms other than GERD, including early satiety, bloating, post-prandial fullness, epigastric pain, epigastric burning, chest pain, nausea, vomiting and chest burning.

### BRIEF DESCRIPTION OF THE DRAWINGS

FIG. 1a illustrates one suitable arrangement for implanting one embodiment of a gastro-electric stimulation system of the present invention;

FIG. 1b shows illustrative components of one embodiment of a gastro-electric stimulation system of the present invention;

FIG. 1c shows an illustrative stimulator and associated medical electrical leads according to one embodiment of the present invention;

FIG. 2a shows a block diagram of one embodiment of an open-loop gastro-electric stimulation system of the present invention;

FIG. 2b shows a block diagram of one closed-loop embodiment of a gastro-electric stimulation system of the present invention;

FIG. 2c shows a block diagram of another embodiment of a closed loop gastro-electric stimulation system of the present invention;

FIG. 2d shows a signal amplitude vs. time chart obtained in accordance with the present invention;

FIG. 3 shows a block diagram of one embodiment of the present invention;

FIG. 4a shows one embodiment of a gastrointestinal stimulation system of the present invention;

FIGS. 4b through 4f illustrate various embodiments of medical electrical leads suitable for use in the system of the present invention;

FIG. 5 illustrates cross-sectional views of various portions of a patient's stomach anatomy;

FIGS. 6a through 6g illustrate various electrode locations in or near the stomach and/or vagus nerve of a patient that may be stimulated and/or sensed in accordance with several embodiments of the present invention;

FIG. 7 illustrates various locations in or near the stomach and/or vagus nerve of a patient for feedback control sensors according to some embodiments of closed-loop feedback control systems of the present invention;

FIGS. 8a through 8c illustrate stimulation pulse, regime and control parameters according to some embodiments of the present invention;

FIG. 9 illustrates several methods of stimulating a patient's stomach and/or vagus nerve so as to minimize, reduce or eliminate GERD symptoms in a patient; and

FIG. 10 shows clinical study data from a WAVESS study obtained in accordance with one embodiment of the present invention;

The drawings are not necessarily to scale. Like numbers refer to like parts or steps throughout the drawings.

### DETAILED DESCRIPTIONS OF THE EXEMPLARY EMBODIMENTS

In the following descriptions of the exemplary embodiments, reference is made to the accompanying drawings that form a part hereof, and in which are shown by way of illustration several specific embodiments of the invention. It is to be understood that other embodiments of the present invention are contemplated and may be made without departing from the scope or spirit of the present invention. The following detailed description, therefore, is not to be taken in a limiting sense. Instead, the scope of the present invention is to be defined in accordance with the appended claims.

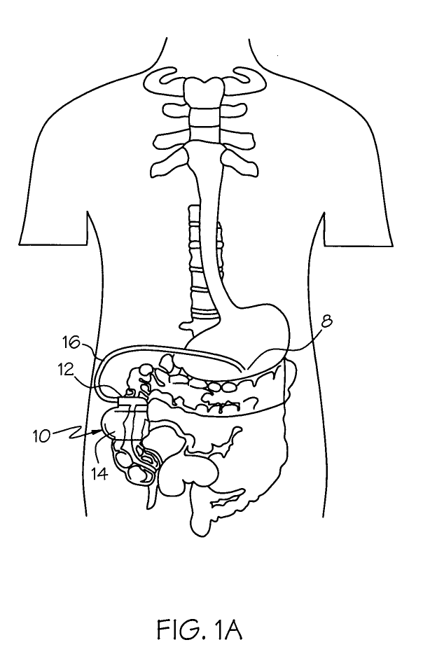

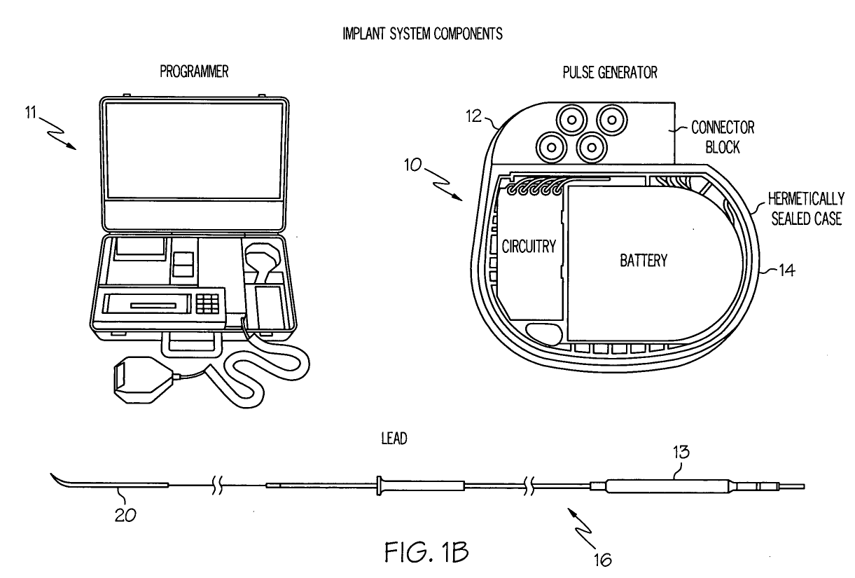

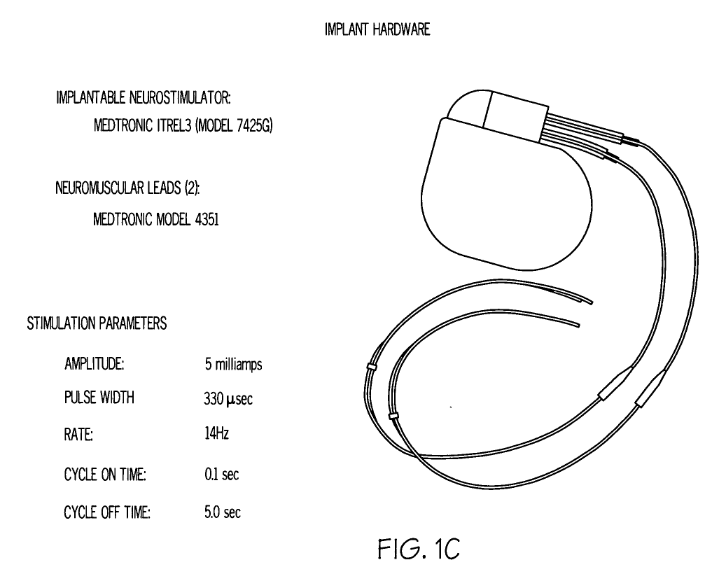

FIG. 1 shows the general environment of a gastro-electric stimulation system of the present invention. The patient depiction shows an abdomen, a digestive system, a stomach, a duodenum, an intestine, a pancreas, an enteric nervous system, and a vagus nerve. The gastro-electric stimulation system may be implanted, or may be located outside the patient. A programmer, separate from the gastro-electric stimulation system, may be used to modify parameters of the gastro-electric stimulation system. Programming may be accomplished with a console remote programmer such as a Model 7432 and Model 7457 memory module software or with a hand-held programmer such as an Itrel EZ, available from Medtronic, Inc. of Minneapolis, Minn.

Nerve impulses generated by electrical stimulation of appropriate portions of the vagus nerve and/or digestive system travel by means of both afferent and efferent pathways to cells in stomach lining which produce gastric acid. Some impulses may travel from the digestive system along a vagal afferent pathway to the brain and then along a vagal efferent pathway from the brain to the stomach lining. Various portions of the stomach are well suited for stimulation in accordance with some embodiments of the present invention. For example, the wall of the stomach is suitable for making electrical connections, and the stomach is well innervated by the vagus nerve. The stomach pacemaker region is particularly well innervated by the vagus nerve.

FIG. 1a further shows one embodiment of INS 10 of the present invention having a lead positioned near a desired or target nerve or nerve portion 8. INS 10 shown in FIG. 1a is a implantable electrical stimulator comprising at least one implantable medical electrical lead 16 attached to hermetically sealed enclosure 14, lead 16 being implanted near desired or target location 8. Enclosure 14 is formed of a biocompatible material such as an appropriate metal alloy containing titanium. It is important to note that at least one more lead 18 (not shown in the drawings) may be employed in accordance with certain embodiments of the present invention, where multiple nerve target sites or portions are to be stimulated simultaneously or sequentially and/or where such multiple target sites or portions are incapable of being stimulated, or are difficult to stimulate, using a single lead even if the single lead contains multiple stimulation electrodes or arrays of stimulation electrodes. FIG. 1c shows an illustrative stimulator and associated medical electrical leads according to one embodiment of the present invention.

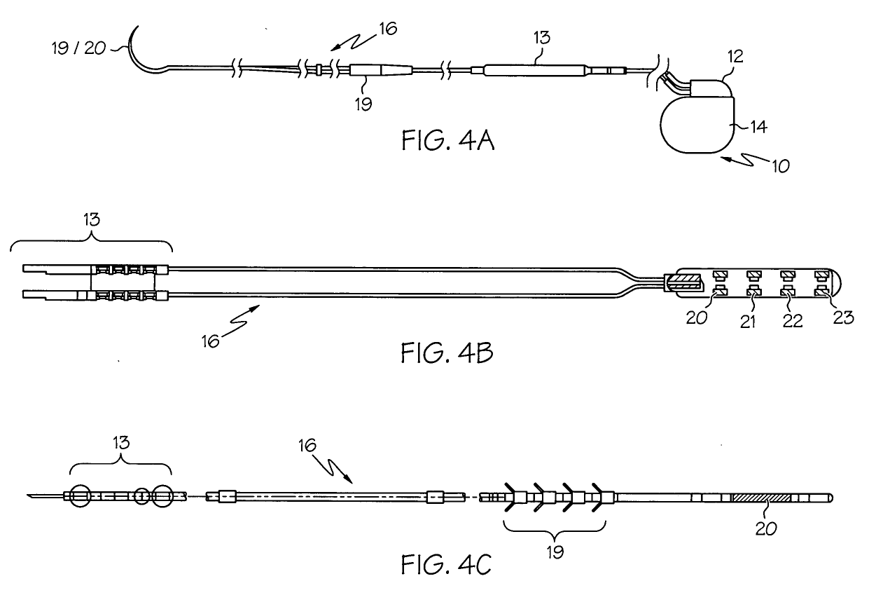

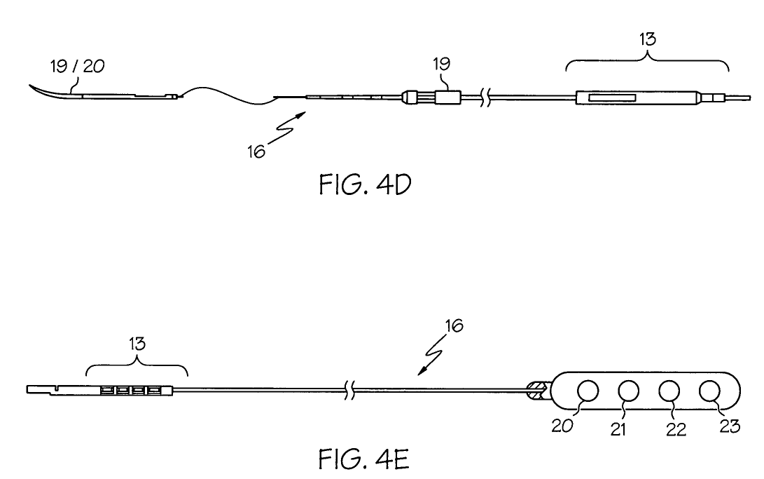

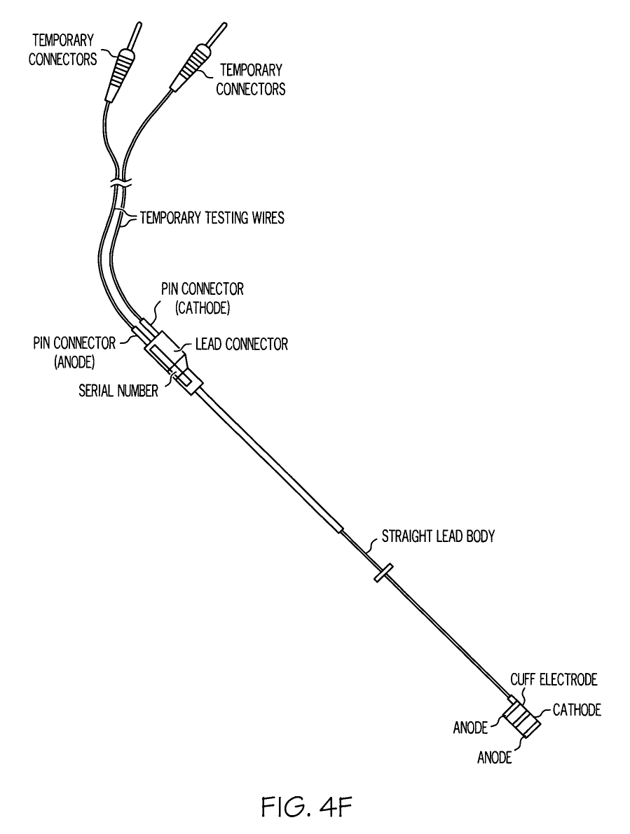

Referring now to FIG. 1b and FIGS. 4a through 4f, lead 16 provides electrical stimulation pulses to the desired nerve target sites or portions and thereby inhibits or excites signals originating in or carried by target stomach tissue, stomach lining, stomach layer, stomach nerve or stomach nerve portion 8 located in the vicinity of the electrode(s) thereof. Leads 16 and lead 18 may have unipolar electrodes disposed thereon (where enclosure 14 is employed as an indifferent electrode) or may have bipolar electrodes disposed thereon, where one or more electrodes disposed on a lead are employed as the indifferent electrode. In one embodiment of the present invention, lead 16 extends from lead connector 13, which in turn forms an integral portion of lead extension 15 connected at its proximal end to connector header module 12.

Leads 16 and 18 are preferably less than about 5 mm in diameter, and most preferably less than about 1.5 mm in diameter. Polyurethane is a preferred material for forming the lead body of leads 16 and 18, although other materials such as silicone may be employed. Electrical conductors extending between the proximal and distal ends of leads 16 and 18 for supplying electrical current to the electrodes are preferably formed of coiled, braided or stranded wires comprising an MP35N platinum-iridium alloy. Electrodes 20, 21, 22 and 23 may be ring electrodes, coiled electrodes, electrodes formed from portions of wire, barbs, hooks, spherically-shaped members, helically-shaped members, or may assume any of a number of different structural configurations well known in the art.

Inter-electrode distances on leads 16 and 18 are preferably about 3 mm, but other inter-electrode distances may be employed such as about 1 mm, about 2 mm, about 4 mm, about 5 mm, about 6 mm, about 7 mm, about 8 mm, about 9 mm, about 10 mm, about 12 mm, about 14 mm, about 16 mm, about 18 mm, about 20 mm, about 25 mm, about 30 mm. Preferred surface areas of electrodes 20, 21, 22 and 23 range between about 1.0 sq. mm and about 100 sq. mm, between about 2.0 sq. mm and about 50 sq. mm, and about 4.0 sq. mm and about 25 sq. mm. Preferred lengths of electrodes 20, 21, 22 and 23 range between about 0.25 mm and about 10 mm, between about 0.50 mm and about 8 mm, and about 1.0 mm and about 6 mm.

The distal portion of lead 16 extends to a desired or target stomach tissue, stomach lining, stomach layer, stomach nerve or stomach nerve portion 8, and is preferably held in such position by lead anchor 19. Note that lead anchor 19 may assume any of a number of different structural configurations such one or more suture sleeves, tines, barbs, hooks, a helical screw, tissue in-growth mechanisms, adhesive or glue.

One, two, three, four or more electrodes 20, 21, 22 and 23 may be disposed at the distal end of lead 16 and/or lead 18. Electrodes 20, 21, 22 and 23 are preferably arranged in an axial array, although other types of arrays may be employed such as inter-lead arrays of electrodes between the distal ends of leads 16 and 18 such that desired or target stomach tissue, stomach lining, stomach layer, stomach nerve or stomach nerve portion 8 disposed between leads 16 and 18 may be stimulated.

Electrode configurations, arrays and stimulation patterns and methods similar to those disclosed by Holsheimer in U.S. Pat. No. 6,421,566 entitled “Selective Dorsal Column Stimulation in SCS, Using Conditioning Pulses,” U.S. Pat. No. 5,643,330 entitled “Multichannel Apparatus for Epidural Spinal Cord Stimulation” and U.S. Pat. No. 5,501,703 entitled “Multichannel Apparatus for Epidural Spinal Cord INS,” the respective entireties of which are hereby incorporated by reference herein, may also be adapted or modified for use in the present invention. Electrode configurations, arrays, leads, stimulation patterns and methods similar to those disclosed by Thompson in U.S. Pat. No. 5,800,465 entitled “System and Method for Multisite Steering of Cardiac Stimuli,” the entirety of which is hereby incorporated by reference herein, may also be adapted or modified for use in the present invention to permit the steering of electrical fields. Thus, although the Figures show certain electrode configurations, other lead locations and electrode configurations are possible and contemplated in the present invention.

Leads 16 and 18 preferably range between about 4 inches and about 20 inches in length, and more particularly may be about 6 inches, about 8 inches, about 10 inches, about 12 inches, about 14 inches, about 16 inches or about 18 inches in length, depending on the location of the site to be stimulated and the distance of INS 10 from such site. Other lead lengths such as less than about 4 inches and more than about 20 inches are also contemplated in the present invention.

Typically, leads 16 and 18 are tunneled subcutaneously between the location of INS 10 and the location or site of target stomach tissue, stomach lining, stomach layer, stomach nerve or stomach nerve portion 8 that is to be stimulated. INS 10 is typically implanted in a subcutaneous pocket formed beneath the patient's skin according to methods well known in the art. Further details concerning various methods of implanting INS 10 and leads 16 and 18 are disclosed in the Medtronic Interstim Therapy Reference Guide published in 1999, the entirety of which is hereby incorporated by reference herein. Other methods of implanting and locating leads 16 and 18 are also contemplated in the present invention.

U.S. patent application Ser. No. 10/004,732 entitled “Implantable Medical Electrical Stimulation Lead Fixation Method and Apparatus” and U.S. patent application Ser. No. 09/713,598 entitled “Minimally Invasive Apparatus for Implanting a Sacral Stimulation Lead ” to Mamo et al., the respective entireties of which are hereby incorporated by reference herein, describe methods of percutaneously introducing leads 16 and 18 to a desired nerve stimulation site in a patient.

Some representative examples of leads 16 and 18 include MEDTRONIC nerve stimulation lead model numbers 3080, 3086, 3092, 3487, 3966 and 4350 as described in the MEDTRONIC Instruction for Use Manuals thereof, all hereby incorporated by reference herein, each in its respective entirety. Some representative examples of INS 10 include MEDTRONIC implantable electrical stimulator model numbers 3023, 7424, 7425 and 7427 as described in the Instruction for Use Manuals thereof, all hereby incorporated by reference herein, each in its respective entirety. See also FIGS. 4b through 4f hereof, which disclose various embodiments of leads 16 and 18 suitable for use in accordance with the present invention. INS 10 may also be constructed or operate in accordance with at least some portions of the implantable stimulators disclosed in U.S. Pat. No. 5,199,428 to Obel et al., U.S. Pat. No. 5,207,218 to Carpentier et al. or U.S. Pat. No. 5,330,507 to Schwartz, each of which is hereby incorporated by reference herein in its respective entirety. Lead locations and electrode configurations other than those explicitly shown and described here are of course possible and contemplated in the present invention.

Referring now to FIGS. 1a through 1c and FIGS. 4a through 4f, leads 16 and 18 may be MEDTRONIC Model 4300 leads, such as the Model 4351 Intramuscular Lead. Leads 16 and 18 are surgically inserted in a patient using a surgical technique such as laparotomy or laparoscopy, with the proximal ends thereof located near INS 10 and the distal ends located near the desired stimulation site 8, such as in the stomach downstream of the lower esophageal sphincter, such as at or adjacent the antrum, or about 10 cm proximal from the pylorus. Alternatively, the distal ends of leads 16 and 18 may be implanted in the “pacemaker region” of the stomach (see FIG. __). Other locations between the downstream end of the lower esophageal sphincter and the pylorus may also be suitable. Leads 16 and 18 may be implanted in a patient percutaneously or inserted with the proximal ends thereof extending outside the patient's body. Leads 16 and 18 should be selected to a peak pulse current of between about 0.01 mA and about 100.0 mA.

According to one method of the present invention, using laparoscopic or laparotomic techniques a surgeon implants leads 16 and 18 into the muscle wall of the antrum 10 centimeters proximal to the pylorus. Intraoperative endoscopy may be used to verify that the placement of electrodes in the stomach wall have not gone full thickness. Electrodes 20-24 are placed 1 cm apart and secured proximally with appropriate anchor mechanisms and distally using small silicone discs and sutures. The proximal connectors of each lead are attached to INS 10, which is implanted subcutaneously in the abdominal wall.

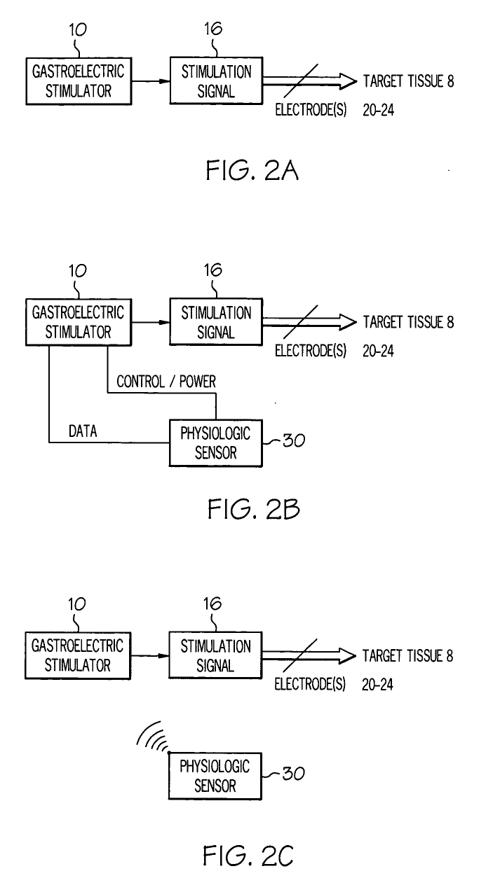

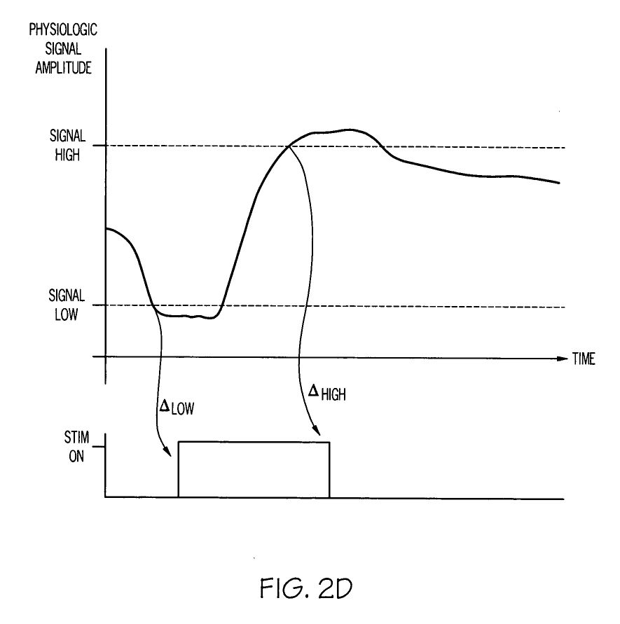

FIG. 2a shows a block diagram of one embodiment of an open-loop gastro-electric stimulation system of the present invention. FIG. 2b shows a block diagram of a closed-loop gastro-electric stimulation system. FIG. 2c shows a block diagram of yet another embodiment of a closed loop gastro-electric stimulation system of the present invention having a wireless connection between physiologic sensor 30 and INS 10.

In a closed-loop embodiment of the present invention, the system is preferably configured such that INS 10 is temporarily disabled so as not to provide electrical stimulation signals to target stomach tissue, stomach lining, stomach layer, stomach nerve or stomach nerve portion 8 after sensor 30 has detected that the patient has swallowed food, by, for example, detecting an increase in pH values. See, for example, U.S. Pat. No. 6,097,984 to Douglas, hereby incorporated by reference herein, in its entirety. Physiologic sensor 30 may be any of a number of suitable sensor types, such as a pH sensor (sensed, for example, either in the esophagus or in the stomach), or any other sensor capable of sensing changes in gastric acidity or pH, changes in the frequency of gastric acid production such as chemical or molecular sensors, a muscle tone sensor (e.g., via pressure manometry with sensors disposed across the lower esophageal sphincter to indicate tightening or relaxation of the muscles thereof), and electrodes for measuring electromyographic activity of the lower esophageal sphincter. The sensed parameter may also be an agonist for gastric acid secretion (e.g., acetylcholine, histamine, gastrin), or may be an antagonist for gastric acid secretion (e.g., prostaglandin, somatostatin, EGF, proglumide).

FIG. 2d shows an illustrative time vs. signal amplitude chart obtained in accordance with the present invention in respect of physiologic sensor 30 and the output signal generated thereby as a function of time. In such a closed-loop feedback control embodiment of the present invention, sensor 30 and sensing and computing circuitry in INS 10 cooperate to detect when a sensed signal has fallen below or risen above a predetermined threshold, as the case may be. Once the sensed signal has remained above or below the predetermined threshold for a predetermined period of time, stimulating circuitry in INS 10 is disabled. Such stimulating circuitry in INS 10 is subsequently enabled or activated when the sensed signal has once again risen above or fallen below the same or a different predetermined threshold.

Some examples of sensor technology that may be adapted for use in some embodiments of the present invention include those disclosed in the following U.S. patents:

U.S. Pat. No. 5,640,764 for “Method of forming a tubular feed-through
hermetic seal for an implantable medical device;”
U.S. Pat. No. 5,660,163 for “Glucose sensor assembly;”
U.S. Pat. No. 5,750,926 for “Hermetically sealed electrical feedthrough
for use with implantable electronic devices;”
U.S. Pat. No. 5,791,344 for “Patient monitoring system;”
U.S. Pat. No. 5,917,346 for “Low power current to frequency converter
circuit for use in implantable sensors;”
U.S. Pat. No. 5,957,958 for “Implantable electrode arrays;”
U.S. Pat. No. 5,999,848 for “Daisy chainable sensors and stimulators
for implantation in living tissue;”
U.S. Pat. No. 6,043,437 for “Alumina insulation for coating implantable
components and other microminiature devices;”
U.S. Pat. No. 6,088,608 for “Electrochemical sensor and integrity tests
therefor;”
U.S. Pat. No. 6,259,937 for “Implantable substrate sensor.”

Each of the foregoing patents is incorporated by reference herein, each in its respective entirety.

In one embodiment of the present invention, physiologic sensor 30 is a motility sensor fixed in, on or near the stomach or esophagus, and provides a stimulation inhibition or disabling signal to INS 10 whenever the patient swallows or exhibits esophageal peristalsis. The inhibiting signal temporarily disables the delivery of stimulation pulses from INS 10 for a duration of time sufficient to permit a sphincter or muscle to relax long enough to allow food or liquid to pass through the esophagus to the stomach.

In still other embodiments of the present invention, an overall therapy aimed at decreasing gastric acid production and/or increasing gastric acid pH may best be delivered by applying a gastric acid secretion “increase signal” for a period of time after a meal has been ingested by a patient. Feedback control algorithms and methods of the present invention may also employ sensing or determining one or more of a patient's rate of gastric acid secretion or production, duodenum salinity, gastric acid impedance, gastric acid electrical activity, motion, pain, weight, nausea, and/or vomiting. As outlined above, such patient conditions may be sensed, measured or determined using an appropriate sensor or sensors that generates a corresponding output signal which is routed to the input of INS 10 for use in controlling electrical stimulation signals. The patient's condition may also be measured by the patient or a physician, who then employs the measured condition to control the electrical stimulation signal output provided by INS 10.

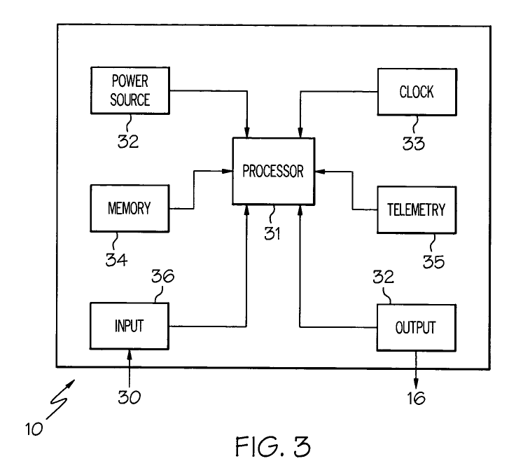

FIG. 3 shows a block diagram illustrating some of the constituent components of INS 10 in accordance with one embodiment of the present invention, where INS 10 has a microprocessor-based architecture. Other architectures of INS 10 are of course contemplated in the present invention, such as the logic or state machine architecture employed in the Medtronic Model Number 3023 stimulator. For the sake of convenience, INS 10 in FIG. 3 is shown with only one lead 16 connected thereto; similar circuitry and connections not shown in FIG. 2 apply generally to lead 18 and other additional leads not shown in the drawings. INS 10 in FIG. 3 is most preferably programmable by means of external programming unit 11 shown in FIG. 1b. One such programmer is the commercially available Medtronic Model No. 7432 programmer, which is microprocessor-based and provides a series of encoded signals to INS 10, typically through a programming head which transmits or telemeters radio-frequency (RF) encoded signals to INS 10. Another suitable programmer is the commercially available Medtronic Model No. 8840 programmer, which is also microprocessor-based but features a touch control screen. Any of a number of suitable programming and telemetry methodologies known in the art may be employed so long as the desired information is transmitted to and from the implantable electrical INS 10.

As shown in FIG. 3, INS 10 receives input signals via physiologic sensor 30, and delivers output stimulation signals to lead 16. INS 10 most preferably comprises a CPU, processor, controller or micro-processor 31, power source 32 (most preferably a primary or secondary battery), clock 33, memory 34, telemetry circuitry 35, input 36 and output 37. Electrical components shown in FIG. 3 may be powered by an appropriate implantable primary (i.e., non-rechargeable) battery power source 32 or secondary (i.e., rechargeable) battery power source 32. INS 10 may also contain a battery or capacitor which receives power from outside the body by inductive coupling between an external transmitter and an implanted receiver. For the sake of clarity, the coupling of power source 32 to the various components of INS 10 is not shown in the Figures. An antenna is connected to processor 31 via a digital controller/timer circuit and data communication bus to permit uplink/downlink telemetry through RF transmitter and receiver telemetry unit 35. By way of example, telemetry unit 35 may correspond to that disclosed in U.S. Pat. No. 4,566,063 issued to Thompson et al. It is generally preferred that the particular programming and telemetry scheme selected permit the entry and storage of electrical stimulation parameters. The specific embodiments of the antenna and other telemetry circuitry presented herein are shown for illustrative purposes only, and are not intended to limit the scope of the present invention.

An output pulse generator provides pacing stimuli to the desired nerve or nerve portion through, for example, a coupling capacitor in response to a trigger signal provided by a digital controller/timer circuit, when an externally transmitted stimulation command is received, or when a response to other stored commands is received. By way of example, an output amplifier of the present invention may correspond generally to an output amplifier disclosed in U.S. Pat. No. 4,476,868 to Thompson, hereby incorporated by reference herein in its entirety. The specific embodiments of such an output amplifier are presented for illustrative purposes only, and are not intended to be limiting in respect of the scope of the present invention. The specific embodiments of such circuits may not be critical to practicing some embodiments of the present invention so long as they provide means for generating an appropriate train of stimulating pulses to the target stomach tissue, stomach lining, stomach layer, stomach nerve or stomach nerve portion 8.

In various embodiments of the present invention, INS 10 may be programmably configured to operate so that it varies the rate at which it delivers stimulating pulses to target stomach tissue, stomach lining, stomach layer, stomach nerve or stomach nerve portion 8 in response to one or more selected outputs being generated. INS 10 may further be programmably configured to operate so that it may vary the morphology of the stimulating pulses it delivers. Numerous implantable electrical stimulator features and functions not explicitly mentioned herein may be incorporated into INS 10 while remaining within the scope of the present invention. Various embodiments of the present invention may be practiced in conjunction with one, two, three or more leads, or in conjunction with one, two, three, four or more electrodes.

It is important to note that leadless embodiments of the present invention are also contemplated, where one or more stimulation and/or sensing electrode capsules or modules are implanted at or near a desired nerve stimulation site, and the capsules or modules deliver electrical stimuli directly to the site using a preprogrammed stimulation regime, and/or the capsules or modules sense electrical or other pertinent signals. Such capsules or modules are preferably powered by rechargeable batteries that may be recharged by an external battery charger using well-known inductive coil or antenna recharging means, and preferably contain electronic circuitry sufficient to permit telemetric communication with a programmer, to deliver electrical stimuli and/or sense electrical or other signals, and to store and execute instructions or data received from the programmer. Examples of methods and devices that may be adapted for use in the wireless devices and methods of the present invention include those described in U.S. Pat. No. 6,208,894 to Schulman et al. entitled “System of implantable devices for monitoring and/or affecting body parameters;” U.S. Pat. No. 5,876,425 to Schulman et al. entitled “Power control loop for implantable tissue stimulator;” U.S. Pat. No. 5,957,958 to Schulman et al. entitled “Implantable electrode arrays;” and U.S. patent application Ser. No. 09/030,106 filed Feb. 25, 1998 to Schulman et al. entitled “Battery-Powered Patient Implantable Device,” all of which are hereby incorporated by reference herein, each in its respective entirety.

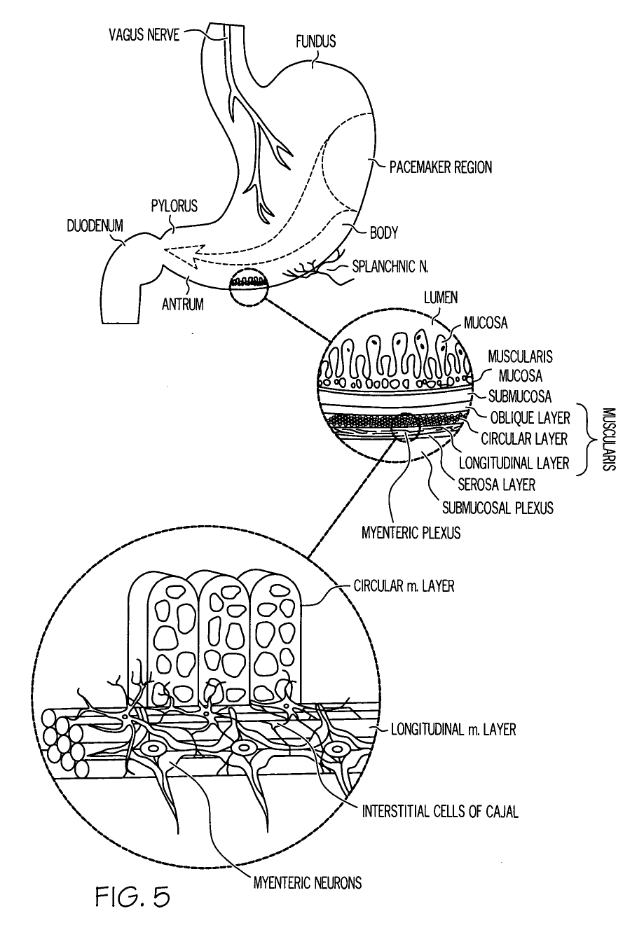

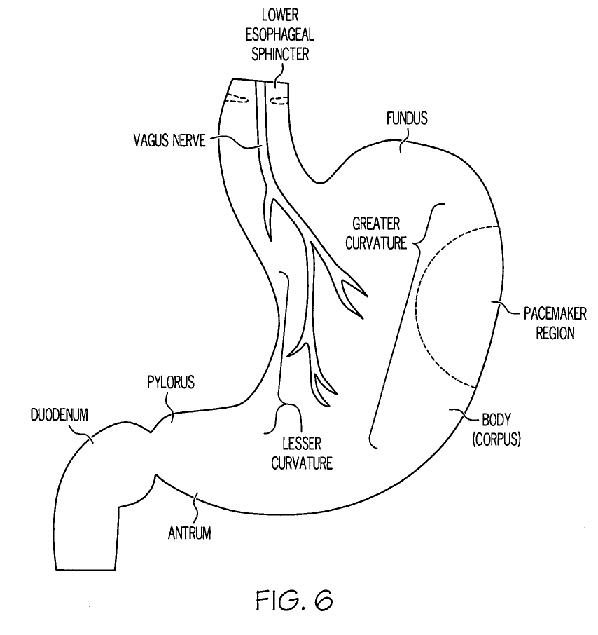

FIG. 4a illustrates one embodiment of an implantable gastro-electric stimulation system suitable for use in the present invention, where the system comprises INS 10 and at least one associated medical electrical lead 16. INS 10 may be an implantable pulse generator (IPG) such as a MEDTRONIC ITREL® 3 Model 7425 implantable stimulator, that produces or generates an electrical stimulation signals adapted for the purposes of the present invention. INS 10 may be surgically implanted such as in a subcutaneous pocket in the abdomen or positioned outside the patient. When positioned outside the patient, the INS 10 may be attached to the patient. INS 10 may be programmed to modify parameters of the delivered electrical stimulation signal such as frequency, amplitude, and pulse width in accordance with various embodiments of the present invention. By way of example, one or more leads 16 and 18 may be implanted into the muscle wall of the stomach such that lead electrodes 20 through 24 of adjacent leads are between about 0.5 cm apart to about 10.0 cm apart, and are located downstream from the lower esophageal sphincter. Particularly preferred locations for stimulation electrodes 20 through 24 are the antrum, body, corpus, lesser curvature, greater curvature and pacemaker region of the stomach as illustrated in FIG. 5, all such location being situated downstream from the lower esophageal sphincter and upstream from the pylorus.

FIGS. 4b through 4f show various embodiments of the distal end of lead 16 of the present invention. In FIGS. 4b and 4e, lead 16 is a paddle lead where electrodes 20-23 are arranged along an outwardly facing planar surface. Such a paddle lead is preferably employed to stimulate peripheral nerves. In FIG. 4c, lead 16 is a conventional quadrapolar lead having no pre-attached anchoring mechanism where electrodes 20-23 are cylindrical in shape and extend around the circumference of the lead body. In FIG. 4d, lead 16 is a quadrapolar lead having tined lead anchors. The tines may be formed from flexible or rigid biocompatible materials in accordance with the application at hand. Representative examples of some tined and other types of leads suitable, adaptable or modifiable for use in conjunction with the systems, methods and devices of the present invention include those disclosed in U.S. patent application Ser. Nos. 10/004,732 entitled “Implantable Medical Electrical Stimulation Lead Fixation Method and Apparatus” and 09/713,598 entitled “Minimally Invasive Apparatus for Implanting a Sacral Stimulation Lead ” to Mamo et al., and those disclosed in U.S. Pat. No. 3,902,501 to Citron entitled “Endocardial Lead,” U.S. Pat. No. 4,106,512 to Bisping entitled “Transvenously Implantable Lead,” and U.S. Pat. No. 5,300,107 to Stokes entitled “Universal Tined Myocardial Pacing Lead.” In FIG. 4d, lead 16 is a quadrapolar lead having a pre-attached suture anchor. In FIG. 4e, lead 16 comprises needle anchor/electrode 19/20 disposed at its distal end and suture anchor 19.

FIG. 4f shows lead 16 as a tri-polar cuff electrode, where cuff/anchor 19 is wrapped around desired nerve or nerve portion 8 to thereby secure the distal end of lead 16 to the nerve and position electrodes 20-22 against or near nerve or nerve portion 8. The Medtronic Model No. 3995 cuff electrode lead is one example of a lead that may be adapted for use in the present invention, the Instructions for Use manual of which is hereby incorporated by reference herein in its entirety.

FIG. 5 illustrates a representative cross-sectional view of gross and microscopic portions of a patient's stomach. The proximal stomach is the fundus and the distal stomach is the body and antrum. The pyloric sphincter joins the antrum and the duodenum. Parasympathetic input to the stomach is supplied by the vagus nerve and the sympathetic nervous system innervates the stomach through the splanchnic nerves. On the greater curvature of the stomach between the fundus and the body is the general region of the pacemaker of the stomach. A telescoped and cross-sectional view of the antrum is shown in the circle in the middle of FIG. 5. This view shows the gastric wall with the mucosal layer and the muscularis. The outermost muscle layer is the longitudinal layer; and running perpendicular to the longitudinal muscle layer is the circular muscle layer. There is also an oblique muscle layer in the stomach. Between the circular muscle and longitudinal muscle layers are neurons of the myenteric plexus and the enteric nervous system. The second telescoped view shown in the lower circle illustrates the anatomic proximities of the myenteric neurons and the interstitial cells of Cajal in the myenteric region between the circular and longitudinal muscle layers. The processes of the interstitial cells interdigitate with circular muscle fibers and the myenteric neurons. The interstitial cells in the myenteric plexus area are thought to be responsible for generation of slow waves or pacesetter potentials. The interstitial cells are also found in the submucosal layers, the deep musculatures plexus, and the intramuscular layers of the stomach. Leads 16 and 18 and electrodes 20-24 may be implanted in or in the vicinity of any one or more of the serosa layer, the myenteric plexus, the submucosal plexus, or any of the various layers of the muscularis (i.e., the oblique, circular or longitudinal layers), but in all cases downstream from or below the lower esophageal sphincter.

In accordance with several embodiments of the present invention, FIG. 5 illustrates various locations for the placement of stimulation and sensing electrodes in and near the stomach, but downstream from the lower esophageal sphincter. Electrodes 20 through 24 are placed in electrical contact or in proximity to target stomach tissue, stomach lining, stomach layer, stomach nerve or stomach nerve portion 8. The electrode location is selected based upon the obtained innervation of the vagus nerve and digestive system, the selected location's suitability for electrode connection, and the degree to which the location proves efficacious for treating GERD in a particular patient. Locations most suitable for electrode attachment and connection should be easily accessible by surgical or endoscopic means, and further be sufficiently mechanically robust and substantial to secure and retain electrodes 20-24 of leads 16 and/or 18.

Continuing to refer to FIG. 5, some specific electrode locations of the present invention that are situated downstream from the lower esophageal sphincter, are well innervated, and surgically or endoscopically accessible include, but are certainly not limited to: (a) the lesser curvature of the stomach; (b) the greater curvature of the stomach; (c) the pacemaker region of the stomach; (d) the antrum of the stomach; (e) the muscularis of the stomach and any of the individual muscle layers comprising the muscularis (i.e., the longitudinal, circular and oblique muscle layers of the stomach); (f) the myenteric plexus of the stomach; (f) the submucosal plexus of the stomach; (g) the serosa layer of the stomach; (h) the muscularis mucosa of the stomach; and (i) the mucosa of the stomach; 0) portions or branches of the vagus nerve extending into or near the stomach, but downstream from the lower esophageal sphincter. Note that as discussed herein, in the present invention it is contemplated that multiple leads and electrodes be employed.

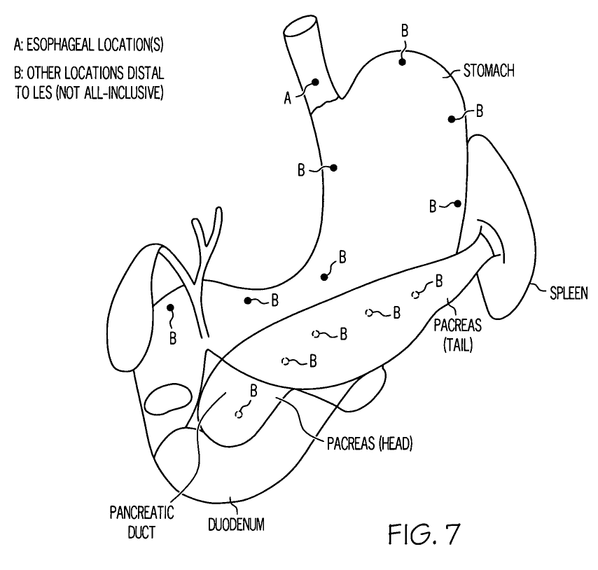

FIG. 7 illustrates various locations in or near the stomach of a patient for feedback control sensors according to some embodiments of closed-loop feedback control systems of the present invention.

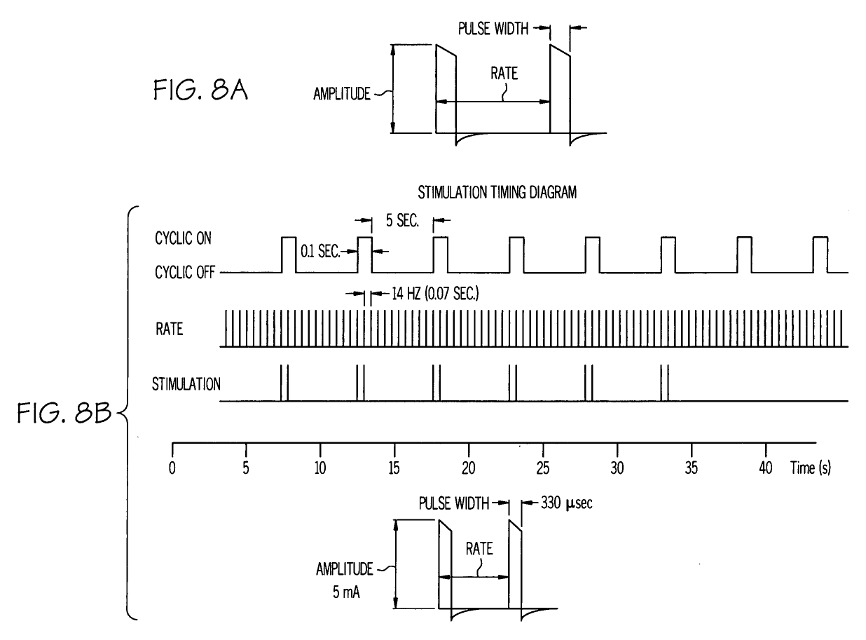

FIGS. 8a through 8c illustrate various representative electrical stimulation pulse, regime and control parameters according to some embodiments of the present invention. FIG. 8a illustrates a typical charge balanced square pulse used in many implantable electrical stimulation systems. As shown, amplitude, pulse width, and pulse rate are adjustable. FIG. 8b shows a timing diagram illustrating the output of INS 10 when the output signal provided thereby successively gated on and off. In FIG. 8b, INS 10 is set to a frequency of 14 pulses per second, but is gated on for 0.1 seconds, and off for 5 seconds, resulting in an output of two pulses every five seconds. The on and off gating periods may be adjusted over a wide range.

In the present invention, electrical stimulation signal parameters may be selected to reduce or eliminate the symptoms attendant to GERD experienced by a patient through direct stimulation of target stomach tissue, stomach lining, stomach layer, stomach nerve or stomach nerve portion 8, by stimulating afferent nerves or nerve portions, by stimulating efferent nerves or nerve portions, or by stimulating some combination of the foregoing. The electrical stimulation signal is preferably charge-balanced for biocompatibility, and adapted to treat the symptoms of GERD.

In the event multiple signals are employed to stimulate a desired site, the spatial and/or temporal phase between the signals may be adjusted or varied to produce the desired stimulation pattern or sequence. That is, in the present invention beam forming and specific site targeting via electrode array adjustments are contemplated. Examples of lead and electrode arrays and configurations that may be adapted for use in some embodiments of the present invention so as to better steer, control or target electrical stimulation signals provided thereby in respect of space and/or time include those disclosed in U.S. Pat. No. 5,501,703 to Holsheimer; U.S. Pat. No. 5,643,330 to Holsheimer; U.S. Pat. No. 5,800,465 to Thompson; U.S. Pat. No. 6,421,566 to Holsheimer; and U.S. patent application Publication No. 20020128694A1 to Holsheimer.

Some representative or exemplary ranges of preferred electrical pulse stimulation parameters capable of being delivered by INS 10 through leads 16 and 18 include the following:

Frequency: Between about 50 Hz and about 100 Hz;

Between about 10 Hz and about 250 Hz; and

Between about 0.5 Hz and about 500 Hz.

Amplitude: Between about 1 Volt and about 10 Volts;

Between about 0.5 Volts and about 20 Volts; and

Between about 0.1 Volts and about 50 Volts.

Pulse Width: Between about 180 microseconds and about 450 microseconds;

Between about 100 microseconds and about 1000 microseconds; and

Between about 10 microseconds and about 5000 microseconds.

Further exemplary stimulation parameters of the system of the present invention include:

(a) A stimulation signal frequency ranging between:

(i) about 0.10 to about 18,000 pulses per minute;

(ii) about 1 to about 5,000 pulses per minute;

(iii) about 1 to about 1,000 pulses per minute;

(iv) about 1 to about 100 pulses per minute;

(v) about 3 to about 25 pulses per minute;

(b) A stimulation signal pulse width ranging between:

(i) about 0.01 mS to about 500 mS;

(ii) about 0.1 mS to about 100 mS;

(iii) about 0.1 mS to about 10 mS;

(iv) about 0.1 mS to about 1 mS;

(c) A stimulation signal current ranging between:

(i) about 0.01 mA to about 500 mA;

(ii) about 0.1 mA to about 100 mA;

(iii) about 0.1 mA to about 10 mA;

(iv) about 1 mA to 100 mA, and

(v) about 1 to about 10 mA.

(d) A stimulation signal which occurs continuously in accordance with the parameters of (a), (b), and (c) above, or a combination thereof;

(e) A stimulation signal which occurs discontinuously when the system turns on and off, where on and off are defined as a cycle time which may vary between about 1 second and about 60 seconds (for example, on=0.1 seconds, and off=5 seconds; on=1.0 sec and off=4 seconds, and so on; see FIGS. 8b and 8c).

(f) Stimulation signals having morphologies best characterized as (i) spikes, (ii) sinusoidal waves, or (iii) square pulses.

Still further exemplary stimulation parameters include:

(a) a stimulation signal frequency ranging between:

(i) about 0.10 to about 18,000 pulses per minute;

(ii) about 1 to about 5,000 pulses per minute;

(iii) about 1 to about 1,000 pulses per minute;

(iv) about 1 to about 100 pulses per minute;

(v) about 3 to about 25 pulses per minute.

(b) a stimulation signal pulse width ranging between:

(i) about 0.01 mS to about 500 mS;

(ii) about 0.1 mS to about 100 mS;

(iii) about 0.1 mS to about 10 mS;

(iv) about 0.1 mS to about 1 mS.

(c) a stimulation signal peak amplitude ranging between:

(i) from about 0.01 mA to about 500 mA;

(ii) from about 0.1 mA to about 100 mA;

(iii) from about 0.1 mA to about 10 mA;

(iv) from about 1 mA to 100 mA, and

(v) about 1 to about 10 mA.

Yet another embodiment of the present invention is a method for treating gastro-esophageal reflux in a patient. The method generally comprises diagnosing gastro-esophageal reflux in a patient; applying at least two electrodes to the stomach of the patient downstream of the lower esophageal sphincter; coupling the electrodes by at least one lead to a neurostimulator; and stimulating the digestive system with a stimulation signal generated by the neurostimulator and conveyed through the lead to the electrodes contacting the stomach of the patient downstream of the lower esophageal sphincter; whereby gastro-esophageal reflux is reduced or eliminated.

In still another embodiment of the present invention, a method of treating gastro-esophageal reflux in a patient generally comprises diagnosing gastro-esophageal reflux in a patient; generating stimulus pulses; and delivering the stimulus pulses to the stomach downstream from the lower esophageal sphincter. Such therapy may be effective for cases of severe reflux disease when drug therapies are ineffective. Compared to fundoplication, GES is relatively less invasive, non-ablative, and reversible. It is anticipated that the side effects and complications from GES will be fewer compared to fundoplication. Fundoplication sometimes results in damage to the vagus nerve, which in turn may cause gastroparesis. Gastric stimulation techniques of the present invention may also be employed to treat symptoms other than GERD, including early satiety, bloating, post-prandial fullness, epigastric pain, epigastric burning, chest pain, nausea, vomiting and chest burning.

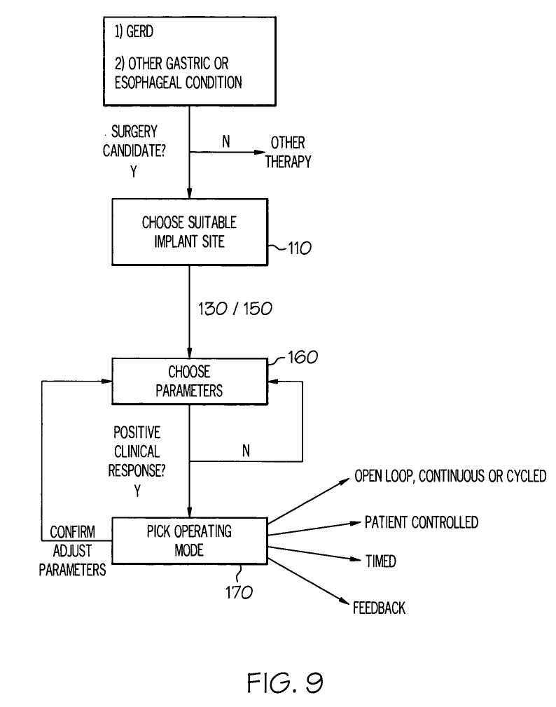

FIG. 9 illustrates several methods of stimulating a patient's stomach so as to treat GERD symptoms in a patient. In FIG. 9, step 110 is employed to determine one or more desired stimulation locations (as illustrated in FIG. 5) positioned near or at one or more of target stomach tissue, stomach lining, stomach layer, stomach nerve or stomach nerve portion 8, as illustrated, for example, in FIG. 5. Next, INS 10 is implanted in step 130 an appropriate location within the patient such that the proximal end of lead 16 may be operably connected thereto and such that INS 10 is placed in such a location that discomfort and the risk of infection to the patient are minimized. Then INS 10 is operably connected to lead 16, which may or may not require the use of optional lead extension 15 and lead connector 13. In Step 150, INS 10 is activated and stimulation pulses are delivered to electrodes 20, 21, . . . n through lead 16 to target stomach tissue, stomach lining, stomach layer, stomach nerve or stomach nerve portion 8. In step 160, the electrical pulse stimulation parameters are adjusted to optimize the therapy delivered to the patient. Such adjustment may entail one or more of adjusting the number or configuration of electrodes or leads used to stimulate the selected location, pulse amplitude, pulse frequency, pulse width, pulse morphology (e.g., square wave, triangle wave, sinusoid, biphasic pulse, tri-phasic pulse, etc.), times of day or night when pulses are delivered, pulse cycling times, the positioning of the lead or leads, and/or the enablement or disablement of “soft start” or ramp functions respecting the stimulation regime to be provided. In step 170 the operating mode of the implanted system is selected. Optionally, parameters selected in step 160 may be adjusted after the operating mode has been selected to optimize therapy.

In the present invention it is also contemplated that drugs be delivered to specific sites within a patient using well known fully implantable drug pump devices in combination with providing electrical stimulation to the nerves or nerve portions described above. According to such a method, the drug pump may be incorporated into the same housing as INS 10, or be separate therefrom in its own hermetically sealed housing. The drug catheter attached to the implantable drug pump through which the drug is delivered to the specific site may also be incorporated into lead 16 or 18, or may be separate therefrom. Drugs or therapeutic agents delivered in accordance with this method include, but are not limited to, antibiotics, pain relief agents such as demerol and morphine, radioactive or radiotherapeutic substances or agents for killing or neutralizing cancer cells, genetic growth factors for encouraging the growth of healthy tissues, and the like.

Incorporated by reference herein in its entirety is U.S. patent application No. 20020082665A1 to Haller et al. published Jun. 27, 2002 and entitled “System and Method of Communicating between an Implantable Medical Device and a Remote Computer System or Health Care Provider.” In the present invention it is contemplated that the methods and devices described herein be extended to include the communication system of Haller et al. for at least one of monitoring the performance of INS 10 and/or an implantable drug pump implanted within the body of a patient, monitoring the health of the patient and remotely delivering an electrical stimulation and/or drug therapy to the patient through INS 10 and/or the optional implantable drug pump, INS 10 or the implantable drug pump being capable of bi-directional communication with a communication module located external to the patient's body, the system comprising: (a) INS 10 and optionally the implantable drug pump; (b) the communication module; (c) a mobile telephone or similar device operably connected to the communication module and capable of receiving information therefrom or relaying information thereto; (e) a remote computer system, and (f) a communication system capable of bidirectional communication.

According to further embodiments of the present invention, an ingestible or implantable pill-shaped or capsular device is employed which is capable of sensing one or more physical parameters such as pH, hormonal levels and the like, and recording, storing or transmitting to an external receiver by, for example, RF means, information regarding the parameter(s) sensed by the device acidity. The sensed parameter information may then be employed to control or refine the gastro-electric stimulation parameters. Examples of devices that may be so adapted in accordance with some embodiments of the present invention include:

U.S. Pat. No. 4,844,076 for “Ingestible Size Continuously Transmitting
Temperature Monitoring Pill” to Lesho et al.;
U.S. Pat. No. 5,170,801 for “Medical Capsule Device Actuated by
Radio-Frequency (RF) Signal” to Casper et al.;
U.S. Pat. No. 5,279,607 for “Telemetry Capsule and Process” to
Schentag et al.;
U.S. Pat. No. 5,395,366 for “Sampling Capsule and Process” to
D'Andrea et al.;
U.S. Pat. No. 6,285,897 for “Remote Physiological Monitoring System”
to Kilcoyne et al.;
U.S. Pat. No. 6,428,469 for “Energy Management of a Video Capsule”
to Iddan et al.;
U.S. patent application Publication No. 20020055734 for “Ingestible
Device” to Houzego et al.;
U.S. patent application Publication No. 20020132226 for “Ingestible
Electronic Capsule” to Nair et al.; and
U.S. patent application Publication No. 20020198470 for “Capsule and
Method for Treating or Diagnosing the Intestinal Tract” to Imran et al..

According to other embodiments of the present invention, implantable sensors and/or stimulation modules or leads may be implanted in desired portions of the gastrointestinal tract by means of a vacuum-operated device which is endoscopically or otherwise emplaced within the gastro-intestinal tract, followed by a portion of the tract being sucked up into a receiving chamber of the device, and the sensor, module or lead being implanted within the tissue held within the receiving chamber. See, for example, U.S. Pat. No. 6,098,629 for “Submucosal Esophageal Bulking Device” to Johnson et al.; U.S. Pat. No. 6,338,345 for “Submucosal Prosthesis Delivery Device” to Johnson et al.; U.S. Pat. No. 6,401,718 for “Submucosal Prosthesis Delivery Device” to Johnson et al.; and PCT Patent Application WO 02087657 for “Gastric Device and Suction Assisted Method for Implanting a Device on a Stomach Wall” to Intrapace, Inc.

In still further embodiments of the present invention, various components of the gastrointestinal electrical stimulation system may be extended, miniaturized, rendered wireless, powered, recharged or modularized into separate or discrete components in accordance with the teachings of, by way of example: U.S. Pat. No. 5,193,539 for “Implantable Microstimulator” to Schulman et al.; U.S. Pat. No. 5,193,540 for “Structure and Method of Manufacture of an Implantable Microstimulator” to Schulman et al.; U.S. Pat. No. 5,324,316 for “Implantable Microstimulators” to Schulman et al.; U.S. Pat. No. 5,358,514 for “Implantable Microdevice With Self-Attaching Electrodes” to Schulman et al.; U.S. Pat. No. 5,405,367 for “Structure and Method of Manufacture of an Implantable Microstimulator” to Schulman et al.; U.S. Pat. No. 5,957,958 for “Implantable Electrode Arrays” to Schulman et al.; U.S. Pat. No. 5,999,848 for “Daisy Chainable Sensors and Stimulators for Implantation in Living Tissue” to Gord et al.; U.S. Pat. No. 6,051,017 for “Implantable Microstimulator and Systems Employing the Same ” to Loeb et al.; U.S. Pat. No. 6,067,474 for “Implantable Device With Improved Battery Recharging and Powering Configuration” to Schulman et al.; U.S. Pat. No. 6,205,361 for “Implantable Expandable Multicontact Electrodes” to Kuzma et al.; U.S. Pat. No. 6,212,431 for “Power Transfer Circuit for Implanted Devices” to Hahn et al.; U.S. Pat. No. 6,214,032 for “System for Implanting a Microstimulator” to Loeb; U.S. Pat. No. 6,315,721 for “System of Implantable Devices for Monitoring and/or Affecting Body Parameters” to Schulman et al.; U.S. Pat. No. 6,393,325 for “Directional Programming for Implantable Electrode Arrays” to Mann et al.; U.S. Pat. No. 6,516,227 for “Rechargeable Spinal Cord Stimulator System” to Meadows et al.

The new treatment for GERD represented by the present invention makes use of gastric electrical stimulation techniques originally devised for the treatment of gastroparesis. The present invention was at least partially conceived of based upon clinical results from a World Wide AntiVomiting Electrical stimulation Study (WAVESS) trial, in which the effectiveness of gastric electrical stimulation (GES) was evaluated in patients suffering from chronic drug refractory gastroparesis. Gastroparesis is a motility disorder characterized by delayed gastric emptying. Patients having gastroparesis are commonly treated with regimen of prokinetic and/or antiemetic drugs. Prokinetic drugs are intended to stimulate smooth muscle contraction and hence improve gastric motility, while antiemetic drugs suppress symptoms of vomiting and nausea.

One of the most commonly used prokinetic drugs, Cisapride (Propulsid), was approved for the treatment of GERD. The majority of patients suffering from gastroparesis have tried this drug at one time or another, although none few or none of them experienced a satisfactory symptomatic response to the medication. Cisapride was withdrawn from the market in the US in June 2000 after problems relating to cardiac arrhythmias surfaced.

Patients enrolled in WAVESS experienced severe symptoms of nausea and vomiting, and it was therefore anticipated that GES would be effective in reducing those symptoms. Clinical results obtained after 12 months demonstrated that GES was indeed effective treating nausea and vomiting. What was not anticipated at the outset of the WAVESS trial was that these same patients experienced severe chest burning symptoms (or heartburn), which is associated with GERD. Surprisingly, it was discovered that symptoms of severe chest burning decreased by statistically significant amounts after 12 months of GES therapy.

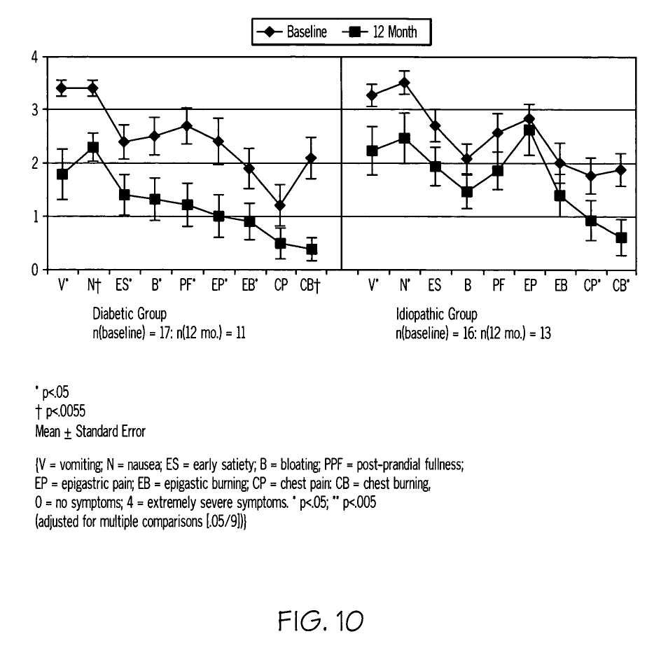

FIG. 10 shows symptom severity profile results at baseline and 12 months from the WAVESS trial. The last symptom in the profile, Chest Burning (“CB”), was statistically improved in patients with diabetic and idiopathic gastroparesis at 12 months in comparison to pre-implant levels. After 12 months of GES therapy, patients on average were nearly symptom free of chest burning.

Thus, electrical stimulation of the stomach at locations situated downstream from the lower esophageal sphincter was discovered to provide a new and novel therapy for the treatment of GERD.

The technique employed in WAVESS and now presently commercialized under Medtronic's therapy name ENTERRA employs two monopolar lead connected to an implantable neurostimulator. The hardware employed in the ENTERRA system is illustrated in FIG. 11. Two electrodes are usually implanted in the muscle wall of the antrum of the stomach about 10 cm above the pylorus. The leads are connected to an implantable neurostimulator which is programmed as follows: amplitude=5 milliamps; pulse width=330 microsecond; frequency=14 hz; cycle on=0.1 sec; cycle off=5 sec. These parameters produce pairs of pulses every five seconds (12 times per minute) which is approximately 4 times the intrinsic frequency of neuromuscular rhythm of the stomach.

It should be mentioned that the specific mechanisms according to which the present invention suppresses symptoms of GERD remain largely unknown at the present time. Some possible mechanisms of the present invention include improved smooth muscle contraction (i.e., improved gut motility), regularization of the gastric slow wave (which is known to be irregular in many gastroparetic patients), or a central nervous system mechanism mediated via a vagal afferent pathway. The present invention may also work by suppressing gastric acid production. Regularization of the gastric slow wave seems most likely since the gastro-esophageal sphincter does not cause a consistent improvement in gastric emptying. Similarly, unpublished reports indicate that not all patients with gastroparesis have irregular gastric electrical activity to begin with. Other reports have shown that GES can affect heart rate variability, which is consistent with a vagal afferent mechanism of action.

The present invention may also work by improving the tone of the muscles in the lower esophageal sphincter. In most cases, gastro-esophageal reflux occurs because the lower esophageal sphincter (LES) opens at inappropriate times, usually because the LES muscles do not clamp down tightly enough to prevent reflux, or because the LES intermittently and spontaneously opens. The LES in a normal healthy patient is normally closed. In at least some embodiments of the present invention, it may be that a patient suffering from GERD is successfully treated by stimulating a portion of the stomach downstream from the LES, which in turn results in an increase in the tone of the LES muscles. Accordingly, the muscles in the LES clamp or contract more tightly and the LES becomes less leaky.

Transient relaxations of the LES muscles may also account for the treatment success enjoyed by the present invention. Such transient relaxations occur spontaneously. Depending on how long the LES muscles remain relaxed or open, and on the contents of the stomach, some reflux may occur. The present invention may work by inhibiting such transient relaxations, or alternatively by exciting contraction of the LES muscles.

As the mechanism of action of the GES on GERD symptoms is not clearly understood at the present time, the techniques described above employed in the WAVESS study or in the ENTERRA system may not be optimal for the treatment of GERD in some or all patients. Depending on the particular circumstances at hand, other stimulation parameters and other lead placement locations may be more effective than those described in above in connection with the WAVESS study or ENTERRA.

Various embodiments of gastric electrical stimulation systems for treatment of GERD have been disclosed herein. One skilled in the art will appreciate that the present invention may be practiced using embodiments other than those specifically disclosed herein, yet remain within the scope of the present invention and the teachings set forth herein. Accordingly, the various embodiments of the present invention disclosed herein are presented for purposes of illustration only, and are not intended to be limiting. Instead, the scope of the present invention is limited only by the claims that follow.

What is claimed is:

1. A system for treating gastro-esophageal reflux disease, comprising:
a pulse generator for generating stimulus pulses; and
a medical electrical lead adapted to deliver the stimulus pulses to the stomach downstream from the lower esophageal sphincter.

2. The system of claim 1, wherein the pulse generator is an implantable neurological stimulator.

3. A gastro-electric stimulator for treating gastro-esophageal reflux disease in a patient, comprising:
a neuro-electrical stimulator for producing a stimulation signal;
at least one electrical lead having a proximal end and a distal end, the proximal end being connected to the neuro-electrical stimulator and the distal end being positionable in a lead position within the patient's stomach and downstream from the lower esophageal sphincter; and,
at least two electrodes carried near the electrical lead distal end, the electrodes being electrically connected through the electrical lead to the neuro-electrical stimulator to receive the stimulation signal and convey such signal to an electrode position adjacent or within the stomach downstream of the lower esophageal sphincter.

4. The gastro-electric stimulator of claim 3, wherein the electrode position is selected from the group consisting of adjacent to or within the antrum of the stomach, the corpus of the stomach, the lesser curvature of the stomach, the greater curvature of the stomach, and the pacemaker region of the stomach.

5. The gastro-electric stimulator of claim 3, wherein the electrode position is upstream from the pylorus.

6. The gastro-electric stimulator of any of claims 3-5, wherein the stimulation signal frequency ranges between about 0.10 pulses per minute and about 18,000 pulses per minute.

7. The gastro-electric stimulator of any of claims 3-5, wherein the stimulation signal pulse width ranges between about 0.01 mS and about 500 mS.

8. The gastro-electric stimulator of any of claims 3-5, wherein the stimulation signal has a peak amplitude ranging between about 0.01 mA and about 500.0 mA.

9. A method for treating gastro-esophageal reflux disease in a patient, comprising:
diagnosing gastro-esophageal reflux disease in a patient;
generating stimulus pulses; and
delivering the stimulus pulses to the stomach downstream from the lower esophageal sphincter in an amount and manner effective to at least reduce the symptoms of gastro-esophageal reflux disease in the patient.

10. The method of claim 9, wherein the step of delivering stimulus pulses further comprises providing at least two electrodes across a portion of the patient's stomach.

11. The method of claim 9, wherein the step of delivering stimulus pulses further comprises positioning the electrodes adjacent or within at least one of the antrum of the stomach, the corpus of the stomach, the lesser curvature of the stomach, the greater curvature of the stomach, and the pacemaker region of the stomach.

12. The method of claim 9, wherein the step of delivering stimulus pulses further comprises positioning the electrodes upstream from the pylorus.

13. The method of any of claims 10-12, wherein the stimulation signal frequency ranges between about 0.10 pulses per minute and about 18,000 pulses per minute.

14. The method of any of claims 10-12, wherein the stimulation signal pulse width ranges between about 0.01 mS and about 500 mS.

15. The method of any of claims 10-12, wherein the stimulation signal has a peak amplitude ranging between about 0.01 mA and about 500.0 mA.

16. A method for treating gastro-esophageal reflux disease in a patient, comprising:
diagnosing gastro-esophageal reflux in a patient;
applying at least one electrode to the stomach of the patient downstream of the lower esophageal sphincter;
coupling the at least one electrode via at least one medical lead to a neurostimulator; and
stimulating the digestive system with a stimulation signal generated by the neurostimulator and conveyed through the lead to the at least one electrode in an amount and manner effective to at least reduce the symptoms of gastro-esophageal reflux disease in the patient.

17. The method of claim 16, wherein the step of delivering stimulus pulses further comprises providing at least two electrodes across a portion of the patient's stomach.

18. The method of claim 16, wherein the step of delivering stimulus pulses further comprises positioning the electrodes adjacent or within at least one of the antrum of the stomach, the corpus of the stomach, the lesser curvature of the stomach, the greater curvature of the stomach, and the pacemaker region of the stomach.

19. The method of claim 16, wherein the step of delivering stimulus pulses further comprises positioning the electrodes upstream from the pylorus.

20. The method of any of claims 17-19, wherein the stimulation signal frequency ranges between about 0.10 pulses per minute and about 18,000 pulses per minute.

21. The method of any of claims 17-19, wherein the stimulation signal pulse width ranges between about 0.01 mS and about 500 mS.

22. The method of any of claims 17-19, wherein the stimulation signal has a peak amplitude ranging between about 0.01 mA and about 500.0 mA.
